(19) United States
(12) Patent Application Publication
(10) Pub. No.: US 2026/0098082 A1
(43) Pub. Date: Apr. 9, 2026

(54) MODULATORS OF NAUSEA AND VOMITING

(71) Applicant: Alberto CHIARUGI, Firenze (IT)

(72) Inventor: Alberto CHIARUGI, Firenze (IT)

(21) Appl. No.: 19/111,781

(22) PCT Filed: Sep. 13, 2023
(86) PCT No.: PCT/IT2023/050207

§ 371 Date: Mar. 13, 2025

(30) Foreign Application Priority Data
Sep. 14, 2022 (IT) 102022000018780

(51) Int. Cl. C07K 16/18; C07K 16/28

(52) U.S. Cl. CPC C07K 16/18; C07K 16/28

(57) ABSTRACT

A method in which anti-Calcitonin gene related peptide (CGRP) monoclonal antibodies are used for direct treatment of nausea and for vomiting, including chemotherapy-induced nausea and vomiting (CINV) induced by multiple triggers. Among them, drugs frequently prompt nausea and vomiting, such as antitumor chemotherapeutics which are among the most emetic drugs and often cause CINV, which in turn can severely compromise efficacy of therapy, as well as patient quality of life. The method uses anti-CGRP monoclonal antibodies to target calcitonin gene related peptide (CGRP), a neuropeptide with pleiotypic effects in the human body, to prevent and counteract CINV.

### TECHNICAL FIELD

The present invention refers to the field of drugs for the treatment of drug-induced nausea and vomiting

### BACKGROUND ART

It is well known that neoplastic patients often suffer from nausea and vomiting. The latter are very frequently associated with the use of chemicals adopted to counteract cancer growth in these patients. Among the different drugs, antineoplastic chemotherapeutics are those most frequently associated with nausea and vomiting, a condition typically defined “chemotherapy induced nausea and vomiting (CINV). CINV is of remarkable clinical relevance because on the one hand compromises the health status of the patient, and on the other limits dosing and causes treatment interruption.

Molecular mechanisms underlying CINV have been in part understood. To trigger nausea and vomiting drugs act at the level of the brainstem, specifically at the area postrema, also known as “chemoreceptor trigger zone”. Here, neuronal populations capable of detecting different toxic xenobiotics present in blood send projections and activate the vomiting center. These response is of fundamental relevance to survival because allows gastric emptying once a given toxic has been ingested. In light of the potent cytotoxic properties of antitumor chemotherapeutics is makes sense that they promptly activate the chemoreceptor trigger zone and causes CINV. The state of the art, therefore, teaches that mechanisms triggering nausea and vomiting during CINV are different from those involved in nausea and/or vomiting prompted by different conditions such as pregnancy, dizziness, pain or anxiety. Indeed, CINV is prompted by the toxic effects of antitumor chemotherapeutics in the gut as well as to their detection by the chemoreceptor trigger zone, whereas the other forms of nausea and vomiting are not due to activation of the chemoreceptor trigger zone.

Given the high incidence of CINV and its remarkable clinical relevance, the identification of efficacious compounds able to prevent or counteract this disorder has received a great deal of attention. In this regard, the state of the art teaches that antiemetics able to counteract vomiting caused by kinetosis (motion sickness), pregnancy, food intoxication, pain or migraine such as scopolamine, metoclopramide, domperidone, meclizine, doxylamine, dimedrinate or Vitamin B6 are not able to counteract CINV. As mentioned above, this inefficacy is due to the fact that the intrinsic cytotoxic profile of antineoplastic chemotherapeutics detected in the area postrema is primarily responsible for the pathogenesis of CINV. In keeping with the specific mechanisms involved in the pathogenesis of CINV, the latter is counteracted by specific drugs such as antagonists of the serotonin 5HT3 receptors (ondansetron, palosetron, granisoetron), antagonists of neurokinin-1 receptors (aprepitant, netupitant) or corticosteroids (dexamethasone). These compounds, indeed, are not used for the treatment of nausea and vomiting prompted by disorders different from CINV. On this basis, it is not obvious to the expert of the field that antiemetics nonspecific for the treatment of CINV can be used to prevent it. Conversely, the expert of the field knows that the state of the art defines guidelines for the treatment of CINV (New Eng. J. Med., 2016, 374, 1356-67) related to the use of antiemetics specific for the therapy of this disorder.

Unfortunately, it is well known that drugs used for the treatment of CINV are not active in some patients and often tend to lose efficacy during treatment cycles. The technical problem to be solved, therefore, is that of identifying new drugs able to act, alone or in cotreatment paradigms, within the brain regions triggering nausea and vomiting in order to improve therapy of CINV and quality of life of neoplastic patients.

In the field of nausea and vomiting, the underlying neurochemistry is still in part obscure. In this regard, a great deal of attention is focused on the identification of neuropeptides regulating signaling among the different brainstem regions involved in nausea and vomiting. Neuropeptides are proteinaceous, small molecules (10-40 amino acids) able to regulate a myriad of neuronal and endocrine functions. Neuropeptides are released by the classic presynaptic apparatus but, at variance with neurotransmitters such as noradrenaline, acetylcholine or serotonin, generate long lasting signaling that can reach regions distant from the presynaptic terminal (the so called “volume transmission”).

Calcitonin gene-related peptide (CGRP) is a 37 amino acid neuropeptide present in the central and peripheral nervous system. The peripheral functions of CGRP are well known and mainly consist in mediating vasodilation and sensitization to pain. In contrast with this, the central functions of CGRP are in large part still unknown. One of the main reasons responsible for this lack of knowledge is the unavailability of brain permeant CGRP receptor agonists and antagonists. Indeed, the current CGRP receptor interacting drugs administered in the periphery are unable to cross the blood brain barrier and regulate CGRP-dependent neurotransmission (Nat. Rev. Neurol., 2018; 14:338). In keeping with this, the expert of the filed knows that to circumvent this pharmacokinetics problem and modulate CGRP neurotransmission the sole strategy is to inject the receptor agonists or antagonists directly into the brain by means of microiontophoresis or intracerebroventricular routes. An additional, recently adopted strategy able to regulate CGRP neurotransmission is the use of viruses that, again directly injected into the brain of experimental animals, carry a genetic information allowing modulation of expression of CGRP or of its cognate receptor. Thanks to these modern gene therapy approaches necessitating direct brain injections, a key role of CGRP in the regulation of neurotransmission among central nervous system regions such as the amygdala, the parabrachial nucleus, the nucleus of the solitary tract, the trigeminal nucleus and several hypothalamic nuclei has recently emerged. However, the expert of the field knows that, just because of the above mentioned impermeability of the blood brain barrier to current CGRP modulating drugs, there is no information in the state of the art regarding the possibility to modulate CGRP neurotransmission in the brain with compounds administered peripherally (i. e, orally, subcutaneously, or intravenously).

A recent advancement of remarkable therapeutic significance is the clinical development of monoclonal antibodies (mAbs) capable of inhibiting peripheral CGRP functions. The mAbs fremanezumab, galcanezumab and eptinezumab bind and scavenge the neuropeptide whereas erenumab binds and inhibits the neuropeptide receptor (these antibodies are collectively defined as “anti-CGRP mAbs”). These mAbs are administered subcutaneously or intravenously and are exclusively efficacious in migraine prophylaxis. A great deal of effort has been devoted to the identification of the mechanisms underlying the antimigraine actions of the anti-CGRP mAbs. The state of the art teaches that the blood brain barrier is impermeable to immunoglobulins (proteins with quaternary structure and molecular weight in the order of 150 kDa). Accordingly, the state of the art teaches that the anti-CGRP mAbs are unable to cross the blood brain barrier and penetrate the brain parenchyma, thereby exerting their pharmacodynamic effects exclusively in the periphery (J Neurosci. 2019; 39:6001-6011; Cephalalgia 2020; 40:229-240; Cephalalgia. 2020; 40:924-934; BMC Neurology, 2022, 22: 205-213). The expert of the field, indeed, ascribes the antimigraine effects of the anti-CGRP mAbs to their peripheral actions. Specifically, it is largely acknowledged that the antimigraine effect of the anti-CGRP mAbs is due to their ability to counteract CGRP-dependent pain signaling at trigeminovascular afferents within the meninges. (Nat. Rev. Neurol. 2018; 14:338-350; CNS Neurol. Disord. Drug. Targets. 2020; 19:344-359).

Hence, the expert of the field does not find any clue in the state of the art that the anti-CGRP mAbs enter and exert functional effects in the central nervous system. A putative brain activity of these antibodies is not obvious because the blood brain barrier is impermeable to immunoglobulins and, reportedly, fremanezumab is unable to reach the brain parenchyma (Cephalalgia 2020; 40:229-240).

Further, the expert of the field does not find any clue in the state of the art that the anti-CGRP mAbs are able to counteract CINV or other types of drug-induced nausea and vomiting. According to the state of the art, this effect is not obvious because antibodies do not cross the blood brain barrier while brain penetrant drugs are necessary to treat drug-induced nausea and vomiting including CINV.

### DISCLOSURE OF THE INVENTION

Unexpectedly, we have now found that the subcutaneous (i.e. peripheral) administration of anti-CGRP mAbs such as fremanezumab, galcanezumab, eptinezumab and erenumab, in contrast with the state of the art (Cephalalgia 2020; 40:229-240), causes an accumulation of said antibodies in the brain. Furter, we have unexpectedly found that the subcutaneous (i.e. peripheral) administration of anti-CGRP mAbs such as fremanezumab, galcanezumab, eptinezumab and erenumab, in contrast with the state of the art (Cephalalgia. 2020; 40:924-934), exerts functional effects in the brain, being able to reduce nausea and vomiting prompted by antitumor chemotherapeutics.

Specifically, we have unexpectedly found that the anti-CGRP antibodies fremanezumab, galcanezumab, eptinezumab and erenumab are present in the brain parenchyma of rats receiving the subcutaneous injection of the antibodies (30 mg/kg in the interscapular region) 7 days before sacrifice. The animals have been transcardially perfused with cold saline for 10 min to eliminate from the brain parenchyma traces of blood containing the antibodies present in plasma. Upon this extensive perfusion, specimens for the brain cortex (a structure beyond the blood brain barrier) and trigeminal ganglion (a structure before the barrier) have been collected and processed for Western blotting. The presence of anti-CGRP mAbs in brain parenchyma has been revealed investigating the presence of human antibodies in the rat brain extracts. Indeed, the possible presence of human antibodies in the rat brain extracts must originate from brain permeation of the subcutaneously injected anti-CGRP antibodies. Specifically, upon electrophoresis of the protein extract and blotting, membranes have been incubated with polyclonal anti-human IgG able to recognize both the heavy and light chains of the anti-CGRP mAbs injected. Membranes have been also incubated with anti-human hemoglobin (as a blood contamination marker) in order to confirm that a complete elimination of blood was obtained upon transcardial rat perfusion. By so doing, we unexpectedly found that the anti-CGRP mAbs were detectable not only in the trigeminal ganglion but also in the brain cortex of the animals in the absence of a concomitant detection of hemoglobin (FIG. 1). This demonstrates that the presence of the human antibodies in the brain parenchyma cannot be ascribed to blood retention within the brain tissue extracts. Hence, the presence of the anti-CGRP mAbs fremanezumab, galcanezumab, eptinezumab and erenumab found in the rat cortex extracts must be ascribed, in contrast with the state of the art (Cephalalgia 2020; 40:229-240), to their unexpected permeation of the blood brain barrier and accumulation in the brain parenchyma.

We have also unexpectedly found that the subcutaneous injection of the anti-CGRP antibodies fremanezumab, galcanezumab, eptinezumab and erenumab reduces nausea and vomiting in rats exposed to antitumor chemotherapeutics. It is known that the induction of nausea by a chemical can be evaluated in rats measuring repetitive mouth opening (the so called “gaping”) (Autonomic Neuroscience, 2006, 129, 36-41). We have therefore evaluated the effects of the antibodies fremanezumab, galcanezumab, eptinezumab and erenumab on gaping prompted in rats by the anticancer drugs cisplatin (6 mg/kg, i.p.) or cyclophosphamide (40 mg/kg, i. p.). These drugs have been injected 15 days after the subcutaneous injection of the anti-CGRP mAbs to allow complete tissue antibody distribution. We have unexpectedly found that the number of gaping events (4 h monitoring) is reduced in animals pretreated with the antibodies fremanezumab, galcanezumab, eptinezumab or erenumab compared with control animals (FIG. 2).

It is known that vomiting can be preclinically induced and evaluated in the shrew (Suncus Murinus). We have unexpectedly found that anti-CGRP mAbs reduce vomiting in the shrew exposed to anticancer chemotherapeutics. Specifically, we have evaluated the effects of the subcutaneous injection of fremanezumab, galcanezumab, eptinezumab and erenumab (100 mg/kg) on vomiting events induced in the shrew by cisplatin (6 mg/kg i.p.) or cyclophosphamide (40 mg/kg i.p.). To allow complete tissue antibody distribution, cisplatin and cyclophosphamide have been administered 15 days after the injection of the anti-CGRP mAbs. We have unexpectedly found that the number of vomiting events (4 h monitoring) is reduced in animals exposed to fremanezumab, galcanezumab, eptinezumab or erenumab compared to controls (FIG. 3).

According to the invention, the anti-CGRP antibodies can be formulated and administered via the intravenous, intraarterial, intramuscular, endonasal and subcutaneous routes for the treatment of nausea and vomiting. The amounts of antibodies to be administered are those commonly adopted for this type of drugs, for instance 10-3000 mg with daily, weekly, or monthly administration.

### BRIEF DESCRIPTION OF DRAWINGS

FIG. 1. Western blotting evaluation of the ability of the anti-CGRP antibodies fremanezumab, galcanezumab, eptinezumab and erenumab to cross the blood brain barrier and accumulate within the brain parenchyma. Figure shows that fremanezumab, galcanezumab, eptinezumab or erenumab (30 mg/kg) are present (with their heavy and light chains) in rat brain cortex 7 days after their subcutaneous injection. As an index of the amount of anti-CGRP antibodies present in the periphery of these animals, the amount of fremanezumab in the rat trigeminal ganglion (TG) is shown. Animals have been transcardially perfused with cold saline to eliminate blood/plasma contamination. The complete removal of blood/plasma is evidenced by the lack of hemoglobin in the tissue extracts. Data indicate that fremanezumab, galcanezumab, eptinezumab and erenumab are able to permeate and accumulate in the brain. In the figure positive controls of fremanezumab and hemoglobin (both 10 ng) are also shown.

FIG. 2. Effects of the anti-CGRP antibodies on nausea induced by cisplatin or cyclophosphamide in the rat. Rats (10 animals/group) have been injected subcutaneously with fremanezumab, galcanezumab, eptinezumab or erenumab (100 mg/kg). After 15 days, animals have been exposed to cisplatin (6 mg/kg) or cyclophosphamide (40 mg/kg) injected i.p. and the number of gaping events evaluated for 4 h as an index of nausea. Rats pretreated with fremanezumab, galcanezumab, eptinezumab or erenumab showed a reduced number of gaping events compared to controls. *p<0.05, **p<0.01 vs Controls, ANOVA and Tukeys post hoc test.

FIG. 3. Effects of the anti-CGRP antibodies on vomiting induced by cisplatin or cyclophosphamide in the shrew. Shrews (10 animals/group) have been injected subcutaneously with fremanezumab, galcanezumab, eptinezumab or erenumab (100 mg/kg). After 15 days, animals have been exposed to cisplatin (6 mg/kg) or cyclophosphamide (40 mg/kg) injected i.p. and the number of vomiting events evaluated for 4 h. Shrews pretreated with fremanezumab, galcanezumab, eptinezumab or erenumab showed a reduced number of vomiting events compared to controls. *p<0.05, **p<0.01 vs Controls, ANOVA and Tukeys post hoc test.

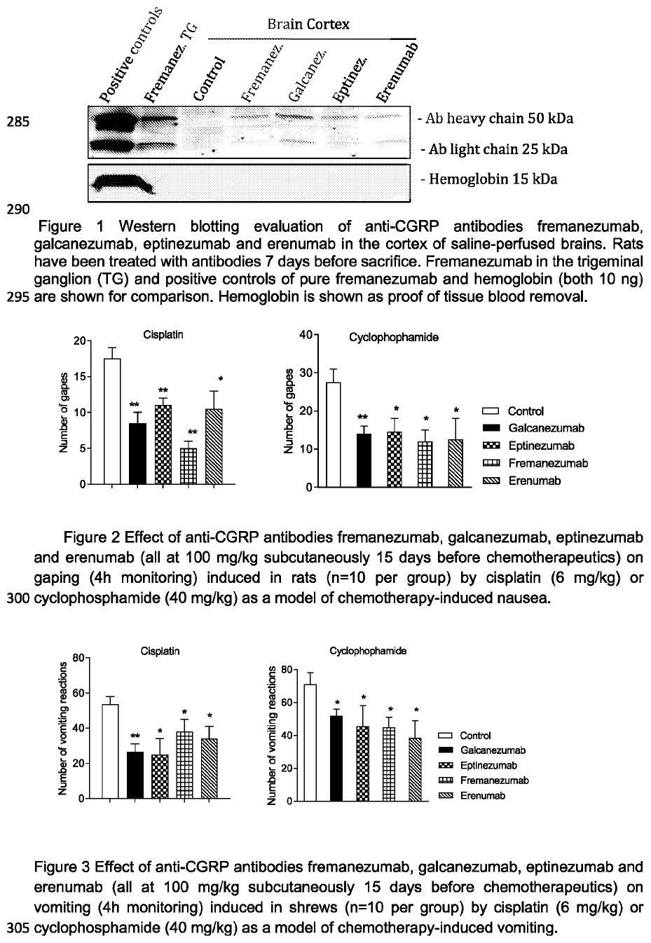

### BEST MODE FOR CARRYING OUT THE INVENTION

The best mode for carrying out the invention is to treat patients before, during and/or after exposure to nausea and/or vomiting inducing agents with daily, weekly or monthly doses of fremanezumab, galcanezumab, eptinezumab or erenumab administered by different routes such as, for example, but not limited to, subcutaneous or intravenous.

1. A method for prevention of chemotherapy-induced nausea and vomiting (CINV) in an individual in need, the method comprising
administering an effective amount of an anti-CGRP antibody, or an anti-CGRP receptor antibody or their antigen-binding fragments in a pharmaceutically acceptable formulation thereof to the individual in need, to prevent chemotherapy-induced nausea and vomiting (CINV) in the individual.

2. A method for treatment of chemotherapy-induced nausea and vomiting (CINV) in an individual in need, the method comprising
administering an effective amount of an anti-CGRP antibody, or an anti-CGRP receptor antibody or their antigen-binding fragments in a pharmaceutically acceptable formulation thereof to the individual in need, to treat chemotherapy-induced nausea and vomiting (CINV) in the individual.

3. A method to potentiate the effect of drugs used to prevent or treat chemotherapy-induced nausea and vomiting in an individual in need, the method comprising
administering an effective amount of an nti-CGRP antibody, or an anti-CGRP receptor antibody or their antigen-binding fragments in pharmaceutically acceptable formulations thereof to the individual in need, to potentiate the effect of drugs used to prevent or treat chemotherapy-induced nausea and vomiting (CINV) in the individual.

4.-6. (canceled)

7. The method of claim 1, wherein said antibody is Fremanezumab.

8. The method of claim 1, wherein said antibody is Galcanezumab.

9. The method of claim 1, wherein said antibody is Eptinezumab.

10. The method of claim 1, wherein said antibody is Erenumab.

11. The method of claim 2, wherein said antibody is Fremanezumab.

12. The method of claim 2, wherein said antibody is Galcanezumab.

13. The method of claim 2, wherein said antibody is Eptinezumab.

14. The method of claim 2, wherein said antibody is Erenumab.

15. The method of claim 3, wherein said antibody is Fremanezumab.

16. The method of claim 3, wherein said antibody is Galcanezumab.

17. The method of claim 3, wherein said antibody is Eptinezumab.

18. The method of claim 3. wherein said antibody is Erenumab.
